(19) United States
(12) Patent Application Publication
(10) Pub. No.: US 2025/0032575 A1
(43) Pub. Date: Jan. 30, 2025

(54) USE OF A MURAMYL PEPTIDE IN THE TREATMENT OF SKELETAL GROWTH RETARDATION

(71) Applicants: UNIVERSITE CLAUDE BERNARD LYON 1, Villeurbanne (FR); CENTRE NATIONAL DE LA RECHERCHE SCIENTIFIQUE, PARIS (FR); ECOLE NORMALE SUPERIEURE DE LYON, LYON (FR); INSTITUTE OF MICROBIOLOGY OF THE CZECH ACADEMY OF SCIENCES, Praha 4 (CZ)

(72) Inventors: François LEULIER, SOLAIZE (FR); Martin SCHWARZER, Nový Hrádek (CZ)

(73) Assignees: UNIVERSITE CLAUDE BERNARD LYON 1, Villeurbanne (FR); CENTRE NATIONAL DE LA RECHERCHE SCIENTIFIQUE, PARIS (FR); ECOLE NORMALE SUPERIEURE DE LYON, LYON (FR); INSTITUTE OF MICROBIOLOGY OF THE CZECH ACADEMY OF SCIENCES, Praha 4 (CZ)

(21) Appl. No.: 18/713,799

(22) PCT Filed: Nov. 28, 2022
(86) PCT No.: PCT/EP2022/083416

§ 371 Date: May 28, 2024

(30) Foreign Application Priority Data
Nov. 29, 2021 (FR) FR2112679

(51) Int. Cl. A61K 38/14; A61K 35/747; A61K 45/06; A61P 19/00

(52) U.S. Cl. CPC A61K 38/14; A61K 35/747; A61K 45/06; A61P 19/00

(57) ABSTRACT

The use of at least one muramyl peptide, such as muramyl dipeptide or an analogue thereof, in the treatment of a skeletal growth retardation in juvenile humans or animals. Also, a probiotic treatment method for promoting skeletal growth in a human or animal, which includes the administration of at least one muramyl peptide and at least one bacterium having intestinal tropism as an adjuvant.

### FIELD

The present invention concerns the use of at least one muramyl peptide, such as muramyl dipeptide or one of its analogues, in the treatment of skeletal growth retardation in a juvenile subject, especially in undernourished humans or animals.

### BACKGROUND

Linear and weight growth is an inherent capacity of all juvenile multicellular organisms. In mammals, postnatal growth is controlled by the somatotropic axis, where growth hormone (GH) instructs the liver and peripheral tissues to produce insulin-like growth factor-1 (IGF-1) in order to promote systemic growth, i.e. the coordinated growth of organs, tissues and the skeleton thereof. Thus IGF-1 is the main factor controlling skeletal growth, also known as linear growth or longitudinal growth. After IGF-1, insulin is the body's main anabolic hormone, regulating carbohydrate, lipid and protein metabolism. While the somatotropic axis (GH/IGF-1) is of paramount importance during growth, coordinated regulation of metabolism by insulin is necessary to provide substrates and fuels for growing tissues. Consequently, insulin also influences the development and growth of animals preferentially via support for weight growth.

Juvenile growth retardation is characterised by a reduced rate of growth during postnatal development. It is manifested by a reduction in weight (weight growth) and a reduction in the height of the individual (skeletal growth, also known as linear growth or longitudinal growth) at a given age compared with the healthy population at the same age. It is a primary manifestation linked to acute undernutrition or chronic under-nutrition and/or recurrent enteric infections, such as diarrhoea and helminthiasis, in early childhood and even before birth, due to malnutrition in the mother during foetal development. In 2012, it was estimated that 162 million children under the age of 5, or 25%, suffered from growth retardation. More than 90% of the world's children suffering from growth retardation live in Africa and Asia, where 36% and 56% of children are affected respectively (United Nations Children's Fund, World Health Organization, The World Bank. UNICEFWHO—World Bank Joint Child Malnutrition Estimates). Once established, growth retardation and its systemic effects generally persist into adulthood and thereafter.

Muramyl peptides are constituents of bacterial cell wall peptidoglycans, which are released into the body during the degradation, growth or division of the bacteria that make up the intestinal microbiota. They are considered natural regulators of immunity. According to numerous studies, muramyl peptides are the minimal biologically active fragments that initiate immune response after interacting with intracellular receptors of the NLR family NOD-like Receptors of innate immunity (NOD1, NOD2, NALP3, etc.). Among these, muramyl dipeptide (MDP, N-acetylmuramyl-L-alanyl-isoglutamine) is a synthetic immunoreactive molecule consisting of N-acetyl-muramic acid appended to a short chain of-Ala-D-isoGln amino acids. It was first identified in the peptidoglycan of the bacterial cell wall as an active component of Freund's complete adjuvant (FCA). In 1974, CDM was found to be the minimum structure required for the efficacy of FCA, one of the most potent and widely used adjuvants in experimental animal models. MDP and its derivatives have been shown to have significant immunomodulatory properties, via one of the PRRs (Pattern Recognition Receptors), the NOD2 receptor (Nucleotide-binding Oligomerization Domain 2) (Girardin S. et al., J. Biol. Chem, 2003, 278(11), p. 8869-72; F. Coulombe et al, 2012 PIoS ONE, 7(5): Article ID e36734). Muramyl dipeptides are also known to activate macrophages and other immune system cells to kill cancer cells (Z. Jakopin, Current Medicinal Chemistry, 2013, 20(16), 2068-2079).

In addition, patent application US 2015/141352 describes the use of muramyl dipeptide (MDP) or a pharmaceutical composition containing it to prevent bone loss, promote bone regeneration and bone formation. Among the bone disorders that can be treated by systemic administration of MDP is especially rickets, a disease of growth and ossification observed in infants and young children in a state of acute malnutrition, mainly due to vitamin D and calcium deficiency. Rickets is characterised by delayed weight growth and insufficient calcification of bones and cartilage.

Although numerous therapeutic applications of muramyl dipeptide or its derivatives have already been provided, its use in the treatment of skeletal growth retardation in chronically undernourished juvenile humans or animals has never been described.

### SUMMARY

The aim of the present invention is actually to re-establish conditions that will enable improved skeletal growth, or even normal skeletal growth (also known as linear or longitudinal growth) to be restored, especially in a context of chronic undernutrition, and in particular in a situation of protein deficiency.

It was in this context that it was surprisingly discovered that muramyl peptides, and in particular muramyl dipeptide (MDP) and its chemical analogues, could be advantageously used, especially by the oral or respiratory route, in the treatment of skeletal growth retardation in juvenile humans or animals, especially when this growth retardation is due to chronic undernutrition, in particular protein deficiency, i.e. a daily protein intake below the recommended dietary allowance (RDA). As a guide, the RDA for protein in children aged 0-36 months is in the order of 9-12 g/day, 15-30 g/day for children aged 4-10 years, 30-50 g/day for boys aged 11-18 years and 30-40g/day for girls aged 11-18 years. It has thus been demonstrated that oral administration of MDP, or one of its analogues such as mifamurtide or murabutide, have a positive effect on skeletal growth (linear or longitudinal) in a model of juvenile mice subjected to a protein-deficient nutritional diet mimicking chronic undernutrition (examples of 1 and 2).

The purpose of the present invention is thus to provide new compositions for improving skeletal growth (linear or longitudinal) in juvenile human or animal subjects, or even restoring normal growth, especially in humans or animals that have been or are being subjected to chronic undernutrition, and in particular protein deficiency.

Another purpose of the invention is to provide such compositions, which can also be used in a therapeutic context.

Yet another objective of the invention is to offer a therapeutic treatment method.

### Objects of the Invention

The first object of the present invention is a composition comprising at least one muramyl peptide, for use in the treatment of skeletal growth retardation (or linear growth or longitudinal growth) in a juvenile human or animal.

According to this first object, the composition may also contain at least one adjuvant for promoting growth, especially juvenile growth, said adjuvant being able in particular to be selected from bacterial strains with intestinal tropism, especially strains of commensal species or species present in the intestinal microbiota of the target species, or acceptable as a probiotic.

Thus, the indication of the composition in accordance with the present invention may be therapeutic or health-related, as a medicinal product or as a pharmaceutical composition, or nutritional, especially as a food supplement, or postbiotic, or even probiotic when said composition contains at least one bacterial strain with intestinal tropism.

A second object of the invention is a method for treating skeletal growth retardation (or linear growth or longitudinal growth) in juvenile humans or animals, comprising administering a composition in accordance with the invention comprising at least one muramyl peptide, optionally in combination with at least one adjuvant for promoting growth, especially skeletal growth to a subject in need thereof.

### Definitions

According to the invention, an analogue or chemical analogue of muramyl dipeptide is a chemical species which differs from muramyl dipeptide only by the replacement of one atom or group of atoms with another, and which has physicochemical and biological properties similar to those of muramyl dipeptide.

The World Health Organisation (WHO) definition of juvenile skeletal growth retardation is that the “height for age” value is less than two standard deviations from the median growth norms for healthy children.

A juvenile subject is one who has not reached sexual maturity.

In this description, the terms “skeletal growth”, “longitudinal growth” and “linear growth” are synonymous with “juvenile growth”. This growth can basically be measured by height in a human or by the size of the snout or mouth at the base of the tail in mammalian animals or by body length in vertebrates and invertebrates.

According to the present invention, “juvenile growth” does not include weight growth, regardless of whether this is related to an increase in muscle mass and/or an increase in fat mass. Conversely, juvenile growth within the meaning of the present invention can be evaluated by an increase in bone length or body length and/or an increase in the production of IGF-1 and/or its functional analogues for animals with an exoskeleton but also devoid of a bony skeleton such as invertebrates, and such as peptides belonging to the family of insulin-like peptides and insulin-like growth factors.

Undernutrition or undernourishment is a state of severe food deprivation, characterised by a food intake that is insufficient to meet an individual's nutritional requirements and daily energy expenditure, leading to nutritional and metabolic deficiencies. Undernutrition can be chronic or acute. In the case of chronic undernutrition, and in particular in the case of protein deficiency, the subjects are in a so-called “stunting” or “short stature” state, i.e. in a state of worsened or even irreversible growth retardation, unlike in the case of acute undernutrition (such as rickets) where the growth retardation, especially in terms of weight, can be reversible.

### BRIEF DESCRIPTION OF THE DRAWINGS

Other features and advantages of the invention will be apparent from the detailed description of the following examples, as well as from the appended FIGS. 1 to 14, which represent the different effects of a treatment on the juvenile growth of young mice treated daily for 5 weeks after weaning with a placebo, MDP or murabutide (Example 1 and FIGS. 1 to 6) or by a placebo or mifamurtide (Example 2 and FIGS. 7 to 12), or by a placebo, MDP alone, a combination of MDP and Lactiplantibacillus plantarum strain WJL bacteria or Lactiplantibacillus plantarum strain WJL bacteria alone (Example 3 and FIGS. 13 and 14) and in which:

FIG. 1: represents the height (in cm) of animals treated with either placebo, CDM or murabutide as a function of the number of days after birth.

FIG. 2: represents the relative height (in %) of animals treated with either placebo, CDM or murabutide as a function of the number of days after birth.

FIG. 3: represents the growth rate (in cm/day) of animals treated with either placebo, CDM or murabutide.

FIG. 4: represents the femur size (in mm) of animals treated with either placebo, MDP or murabutide.

FIG. 5: represents the size of the tibia (in mm) of animals treated with either placebo, MDP or murabutide.

FIG. 6: represents the circulating amount of IGF-1 (in ng/ml) in animals treated with either placebo, MDP or murabutide.

FIG. 7: represents the height (in cm) of animals treated with either placebo or mifamurtide as a function of the number of days after birth.

FIG. 8: represents the relative height (in %) of animals treated with either placebo or mifamurtide as a function of the number of days after birth.

FIG. 9: represents the growth rate (in cm/day) of animals treated with either placebo or mifamurtide.

FIG. 10: represents the femur size (in mm) of animals treated with either placebo or mifamurtide.

FIG. 11: represents the tibia size (in mm) of animals treated with either placebo or mifamurtide.

FIG. 12: represents the circulating amount of IGF-1 (in ng/ml) in animals treated with either placebo or mifamurtide.

FIG. 13: represents the growth gain (in %) in animals treated with either MDP alone, Lactiplantibacillus plantarum strain WJL alone, or MDP+Lactiplantibacillus plantarum strain WJL, in comparison with animals treated with placebo.

FIG. 14: represents the percentage improvement in circulating IGF-1 levels (in %) in animals treated with either MDP alone, Lactiplantibacillus plantarum strain WJL alone, or MDP+Lactiplantibacillus plantarum strain WJL, in comparison with animals treated with placebo.

### DETAILED DESCRIPTION

The first object of the invention is a composition comprising at least one muramyl peptide, for use in the treatment of skeletal growth retardation in a juvenile human or animal.

According to one particular and preferred embodiment of the invention, said human or animal is chronically undernourished. Most preferably, this state of chronic undernutrition is a protein deficiency.

According to one preferred embodiment of the invention, said at least one muramyl peptide is selected from muramyl dipeptide (or MDP or MurNAc-L-Ala-y-D-Glu, CAS No 53678-77-6) and muramyl dipeptide analogues.

MDP is commercially available in freeze-dried form (powder), especially from InvivoGen Europe (Toulouse, France). It is of bacterial origin and can be present in purified or semi-purified form, for example in the form of MDP connected to fragments of bacterial cell wall.

Muramyl dipeptide analogues are synthetic peptides, including especially mifamurtide (or muramyl tripeptide phosphatidyl ethanolamine, also known as MTP-PE) such as, for example, the product sold under the trade name Mepact by the company Takeda, murabutide (or muramyl dipeptide butyl ester) (InvivoGen Europe, Toulouse, France), 6-O-stearoyl-N-acetyl-muramyl-L-alanyl-D-isoglutamine (or L18-MDP), muropeptide MurNAc-Ala-D-isoGln-Ly (M-TriLYS, InvivoGen Europe, Toulouse, France) and mixtures thereof.

According to one preferred embodiment of the invention, the muramyl dipeptide analogue is selected from murabutide and mifamurtide.

According to one preferred embodiment of the invention, said at least one muramyl peptide is muramyl dipeptide.

Said at least one muramyl peptide generally represents from about 10 μg to 100 mg per unit dose of composition, more particularly from about 50 μg to 50 mg, preferably from about 100 μg to 10 mg, and even more preferably from 0.5 to 1 mg per unit dose of composition.

The number of doses to be administered and the frequency of administration of these doses are of course to be adjusted according to the size and weight of the human or animal subject to be treated, as well as the severity of the growth retardation and the bioavailability of the muramyl peptide(s) administered.

According to the present invention, by “unit dose”, it is meant the amount of composition in accordance with the invention likely to be administered, per dose, to a subject to be treated, whatever the total mass of this unit dose.

According to one particular and preferred embodiment of the invention, the composition also comprises at least one adjuvant for promoting growth, especially in a juvenile subject.

According to this particular embodiment, said adjuvant may be selected from bacterial strains with intestinal tropism, bacterial constituents, postbiotics and mixtures thereof.

By bacterium with “intestinal tropism”, it is meant a bacterium with the ability to pass the gastric barrier and which is capable of persisting in the intestines.

In accordance with one advantageous characteristic of the invention, said bacterium can promote production of IGF-1 in humans or animals treated with the composition in accordance with the invention.

Bacterial strains with intestinal tropism that can be used in the composition according to the invention are especially strains of commensal species or species present in the intestinal microbiota of the target species or acceptable as probiotics.

Defined in 2001 by the World Health Organisation (WHO) and the Food and Agriculture Organisation (FAO) of the United Nations, probiotics are living micro-organisms which, when ingested in sufficient amount, exert positive effects on health over and above the usual nutritional effects.

According to the invention, postbiotics are inactivated compounds based on inactivated microbial/bacterial cells or inactivated isolated cellular components (such as short-chain fatty acids, peptides, proteins, bacterial wall components or even enzymes), with or without metabolites, which also offer health benefits.

The bacterial strains are preferably selected from bacteria belonging to the following families: Lactobacillus, Streptoccaceae, Enterococcaceae, Leuconostocaceae, and Bifidobacteriaceae.

Among such strains, particular mention may be made of strains of the genus Lactobacillus, in particular of one of the following species, Lactobacillus delbrueckii, Lactobacillus plantarum (or according to the new name Lactiplantibacillus plantarum), Lactobacillus fermentum, Lactobacillus casei, Lactobacillus paracasei, and Lactobacillus rhamnosus.

More particularly, these are bacteria belonging to the species Lactobacillus plantarum, Lactobacillus fermentum, Lactobacillus casei, Lactobacillus paracasei and Lactobacillus rhamnosus. According to one modality, the bacterial strain is selected from the species Lactobacillus plantarum, Lactobacillus fermentum and Lactobacillus casei.

According to one embodiment, the bacterial strain is selected from L. plantarum WJL deposited with the CNCM (Collection Nationale de Cultures de Micro-organismes—Institut Pasteur) on 15 Feb. 2017 under the number I-5169, L. plantarum IGFL1 deposited on 19 Jul. 2017 with the CNCM under the number I-5217, L. plantarum IGFL2 deposited with the CNCM on 19 Jul. 2017 under number I-5218, L. plantarum G821 deposited with the CNCM on 11 May 2015 under number I-4979, L. plantarum NIZ02877, L. casei ATCC 393, L. casei L919, L. paracasei ATCC25302, L. paracasei Shirota, L. fermentum ATCC9338, L. rhamnosus L900, L. rhamnosus L908, L. rhamnosus GG, and mixtures thereof.

Among such strains, L. plantarum WJL deposited with the CNCM on 15 Feb. 2017 under accession number I-5169, L. plantarum IGFL1 deposited with the CNCM under accession number I-5217 on 19 Jul. 2017, L. plantarum G821 deposited with the CNCM under accession number I-4979 on 11 May 2015, L. plantarum IGFL2 deposited with the CNCM under accession number 1-5218 on 19 Jul. 2017, and L. plantarum NIZ02877 are preferred.

Strain IGFL1 was obtained by experimental evolution (i.e. by selection of natural variants having accumulated mutations naturally induced during DNA replication processes) of strain WJL.

Strain G821 was obtained by experimental evolution (i.e. by selection of natural variants that have accumulated naturally induced mutations during DNA replication) of strain NIZ02877.

Strain IGFL2 was obtained by experimental evolution (i.e. selection of natural variants that have accumulated naturally induced mutations during DNA replication processes) of strain NIZ02877.

Other examples of suitable strains are as follows: L. casei ATCC 393, L. casei L919 (Koryszewska-Baginska A. et al., 26 Sep. 2013, Genome Announc), L. paracasei ATCC25302, L. paracasei Shirota (Yuki N et al., Int J Food Microbiol. on' April 1999; 48(1):51-7), L. fermentum ATCC9338, L. rhamnosus L900 (Aleksandrzak-Piekarczyk T. et al., Genome Announc, 15 Aug. 2013), L. rhamnosus L908 (Koryszewska-Baginska A. et al., 20 Feb. 2014, Genome Announc), L. rhamnosus GG (Kankainen M. et al., Proc Natl Acad Sci USA, 6 Oct. 2009).

The composition according to the invention may comprise the bacterial strain(s) in live form. It may be a bacterial suspension, which can be frozen and thawed before use, or a freeze-dried powder, which can be used as it is or after rehydration in a suitable liquid vehicle. It may then comprise a conventional freeze-drying excipient.

Within the composition in accordance with the present invention, the bacterial strain(s) may be present in an amount of about 10⁵ to 10¹², especially about 10⁶ to 10¹², preferably about 10⁸ to 10¹² colony forming units (CFU), per gram of composition.

The composition according to the invention may also comprise the bacterial strain(s) in inactivated form. The bacterial strains can be inactivated by any suitable technique well known to the person skilled in the art, such as, for example, by bacteriolytic or bacteriostatic chemical treatment or physical treatment by pressure, radiation, heat or ultrasound.

When the composition in accordance with the invention contains, as an adjuvant, bacterial constituents, these may for example be selected from inactivated bacteria, bacterial lysates, purified bacterial walls and fragments of purified bacterial walls of bacteria with intestinal tropism, peptidoglycans and teichoic acids. Bacteria with intestinal tropism that can be used to prepare purified bacterial walls or purified bacterial wall fragments can especially be selected from bacterial strains with intestinal tropism as described above.

According to one particular and preferred embodiment of the invention, the composition comprises purified bacterial walls of L. plantarum WJL and/or purified bacterial wall fragments of L. plantarum WJL.

Within the composition in accordance with the present invention, the bacterial constituent(s) may be present in an amount of from about 0.02 μg to 200 mg, especially from about 0.2 μg to 2 mg, preferably from about 20 μg, per unit dose of composition.

The composition in accordance with the invention may also contain, as an additional ingredient, one or more food supplements and/or nutrients, which may especially be selected from carbohydrates, lipids, peptides other than muramyl peptides, proteins, fatty acids, amino acids such as L-glutamine, vitamins, minerals and prebiotics such as, for example, acacia gum fibres, etc.

The composition in accordance with the present invention may be a form of administration by the oral or respiratory route and may be in the form, for example, of a powder, a capsule or a tablet, a drinkable solution, a nasal spray or an aerial spray. According to one particular embodiment of the invention, said administration form is in an enteric-coated (gastro-resistant) form. This form is particularly suitable when the composition contains at least one bacterial strain as an adjuvant in addition to said at least one muramyl peptide. Indeed, this form enables it to pass through the stomach without being degraded and then release said bacterium in the intestine.

Another object of the present invention is a method for treating skeletal growth retardation in a juvenile human or animal subject, especially in an undernourished subject, said method comprising administering a composition comprising at least one muramyl peptide as defined according to the first object of the invention to a subject in need thereof.

According to one particular preferred embodiment of the invention, the subject is a human or animal subject in chronic undernutrition due to a protein deficiency.

As previously indicated, said method may be a therapeutic treatment or a probiotic treatment (or nutritional supplement) when the composition additionally contains at least one bacterium with intestinal tropism as an adjuvant.

Thus, another object of the present invention is a probiotic or postbiotic treatment method for promoting skeletal growth in an animal or human subject, especially an undernourished subject, said method comprising administering to said subject a composition comprising at least one muramyl peptide and, in the case of probiotic treatment, at least one bacterium with intestinal tropism as an adjuvant, said bacterium being alive or inactivated.

The method in accordance with the invention comprises administering a sufficient amount of a composition in accordance with the first object of the invention and as defined above. The amount and frequency of administration will depend especially on the severity of the skeletal growth retardation and the age and condition of the patient or animal. The method will comprise administering doses of the composition according to the invention on one or more occasions, which may be staggered over the growth period of the subject (up to puberty or sexual maturity). The doses may in particular be divided up to facilitate administration, especially as a function of the subject's age. The frequency of administration is especially between one dose (single or divided) every day and one dose every month. Typically, the frequency of administration will be between one dose (single or divided) every day and one dose every week, or even every 2, 3, 4, 5 or 6 days. Each dose (single or divided) especially represents several grammes to several tens of grammes of composition.

### EXAMPLES

The raw materials used in the examples are as follows:
Muramyl dipeptide sold under the name MDP by the company InvivoGen Europe, 5, rue Jean Rodier, F-31400, Toulouse, France;
Murabutide, sold under the name Murabutide by the company InvivoGen Europe, 5, rue Jean Rodier, F-31400 Toulouse, France;
Mifamurtide, sold under the brand name Mifamurtide by the company CliniSciences, 74 Rue des Suisses, 92000 Nanterre, France;
Dimethylsulfoxide (DMSO), sold under the name Dimethyl sulfoxide by the company Merck, Darmstadt, Germany.
Maltodextrin, sold under the name Maltodextrin by the company Merck, Darmstadt, Germany.
Lactiplantibacillus plantarum strain WJL bacteria (deposited with the CNCM on 15 Feb. 2017 under number 1-5169).

### Example 1: Demonstration of the Effects of Administering Different Muramyl Peptides in a Chronically Undernourished Mouse Model

In this example, the effects of several compositions in accordance with the present invention comprising isomolar amounts of MDP or murabutide (MDP weight-average molar mass (Mw)=492.5 g/mol, murabutide Mw=548.6 g/mol) were tested in a chronically undernourished mouse model, namely:
Composition A comprising 25 μg of MDP in 30 μL of placebo composition,
composition B comprising 28 μg of murabutide in 30 μL of placebo composition.

These compositions have been compared with a placebo composition containing only a cryoprotectant (Maltodextrin) resuspended in 30 μL of sterile saline buffer.

### 1.1 Preparation of the Compositions

15 μg of cryoprotectant have been resuspended in 30 μL of sterile saline buffer to generate a dose of placebo composition.
25 μg of MDP have been resuspended in 30 μL of placebo composition to obtain composition A with a final MDP concentration of 25 μg/30 μL.
28 μg Murabutide have been resuspended in 30 μL of placebo composition for composition B at a final murabutide concentration of 28 μg/30 μL (isomolar to 25 μg/30 μL MDP).

### 1.2 Preparation of Animals

Conventional C57B1/6j laboratory mice have been bred in the Gnotobiology Laboratory for more than 10 generations. Mice have been maintained in IVC cages (Tecniplast, Italy), exposed to 12:12 h light-dark cycles, supplied with tap water and fed ad libitum with sterile diet V1124-300 (Ssniff Spezialdiaten GmbH) (irradiated −25 kGy, Bioster, Czech Republic). The mice have been mated and after delivery, the litter size was reduced to 6 pups per mother. On day 21, male mice have been weaned on the experimental low-protein, low-fat diet (3.5 kcal/gram, protein: 4.1 wt %, carbohydrate: 78 wt %, fat: 2 wt %) and regularly monitored until 56 days after birth (adult age in mice). Mice weaned on the low-protein, low-fat diet have been treated 5 times a week with placebo, composition A containing MDP or composition B containing Murabutide. At the end of the experiment, food and bedding were removed at 8 am and the mice have been sacrificed after 5 hours of fasting by isoflurane inhalation and cervical dislocation. All animals have been sacrificed within one hour between 1 and 2 p.m.

### 1.3 Administration of the Compositions

The daily treatment dose was 30 μL placebo or for MDP 25 μg in 30 μL placebo and for murabutide 28 μg in 30 μL placebo. Each of the compositions has been administered by pipette to the mouse's tongue. The mouse has been maintained until the composition had been swallowed completely.

### 1.4 Description of Tests Performed

To measure body length, the mice have briefly been anaesthetised with isoflurane (Piramal Healthcare, UK). The anaesthetised mouse has been held by the tail and the nose-anus length has been measured using a ruler. Relative length has been calculated as % length gain, where body length at day 21 (weaning) has been set to 100%. The length growth rate has been calculated by dividing the absolute length gain from day 21 to day 56 by the number of days (i.e. 35). Femur and tibia bones have been dissected at sacrifice, fixed in 4% paraformaldehyde—PBS overnight at 4° C., washed with PBS and preserved in 70% ethanol. Bone length has been measured using a digital caliper (Festa).

At the end of the experiment, the blood has been collected and left to coagulate at room temperature for 2 hours. Sera have been separated by centrifugation (2,000×g for 5 minutes, 4° C.) and preserved at −80° C. until use. IGF-1 levels have been measured using the Mouse/Rat IGF-1 Quantikine ELISA kit (R&D systems) according to the manufacturer's instructions. Data have been analysed using the following statistical test: One way ANOVA with Tukey's multiple comparisons test.

### 1.5 Results

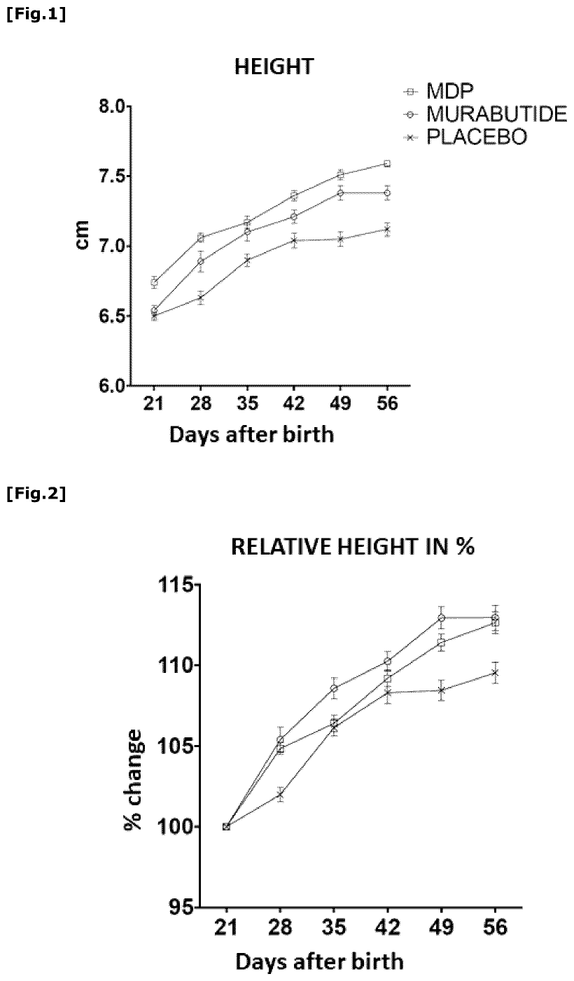

The results obtained are set forth in the appended FIGS. 1 to 6.

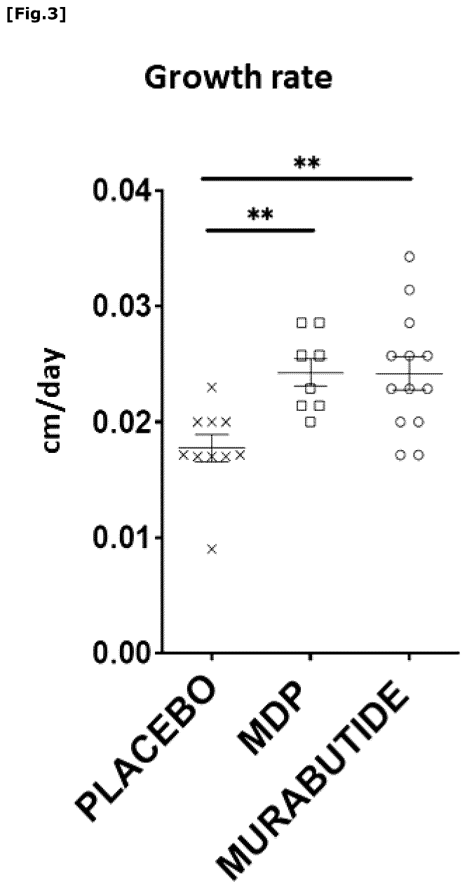

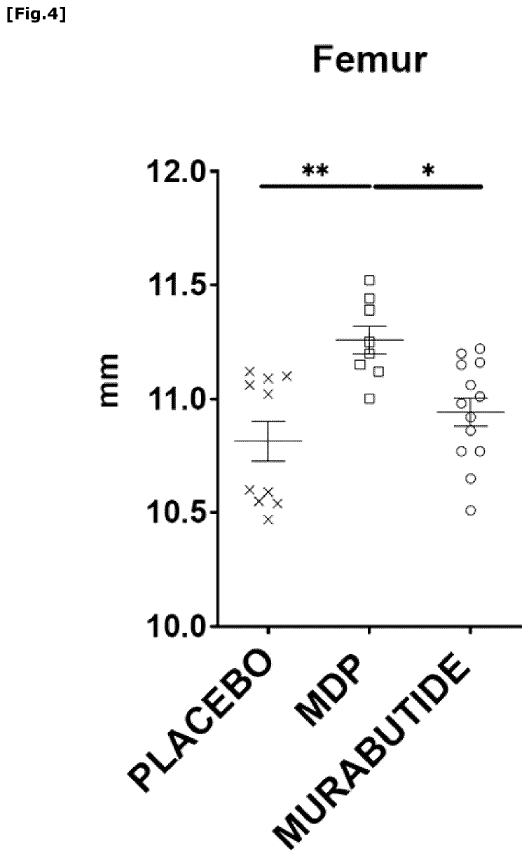

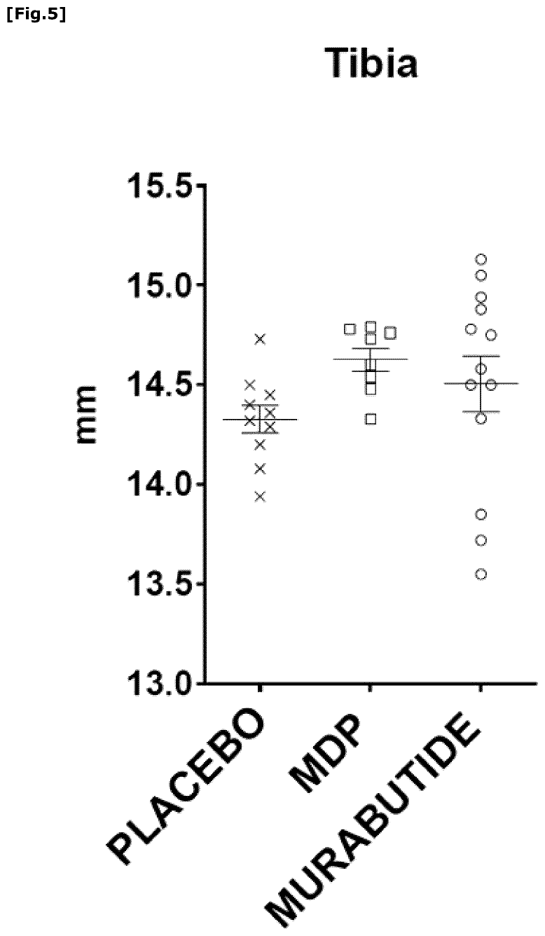

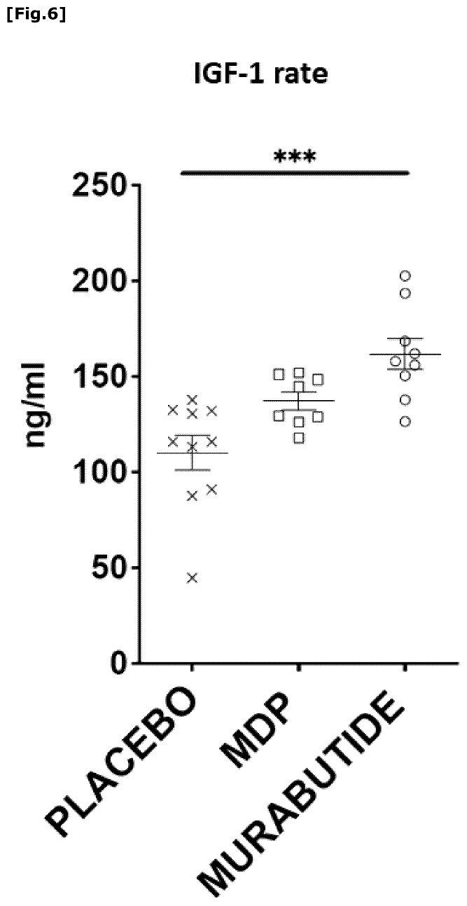

FIG. 1 represents the course of the height (in cm) as a function of the number of days after birth for each of the compositions tested: composition A with MDP: curve with empty squares, composition B with murabutide: empty circles and curve with crosses: placebo composition. FIG. 2 represents the course of relative growth (% change) as a function of the number of days after birth for each of the compositions tested, using the same symbols as for FIG. 1. FIG. 3 represents the course of the growth rate (in cm/day) for each of the compositions tested. FIG. 4 represents the size of the femur (in mm) for each of the compositions tested. FIG. 5 represents the size of the tibia (in mm) for each of the compositions tested. FIG. 6 represents the IGF-1 blood level (in ng/ml) for each of the compositions tested.

These results indicate a significantly increased relative growth, total height and growth rate in mice given composition A comprising MDP or composition B comprising Murabutide compared with mice given the placebo composition. In particular, the femur of animals treated with MDP composition A was significantly larger than that of animals treated with the placebo composition, with a positive trend in murabutide treatment that did not reach statistical significance. The length of the tibia also showed a positive trend of increased growth following the MDP and murabutide treatments compared with the placebo treatments, but this did not reach statistical significance. Finally, the circulating IGF1 level was significantly increased in animals treated with murabutide, and a positive but non-significant trend has been observed in animals treated with MDP compared with animals treated with placebo.

### Example 2: Demonstration of the Effects of Mifamurtide Administration in a Chronically Undernourished Mouse Model

In this example of the present invention, the effects of a composition comprising isomolar amounts of a muramyl peptide mifamurtide (Mw=1237.50 g/mol) were tested in a model of chronically undernourished mice: namely Mifamurtide 62.5 μg/day in 30 μL of placebo.

This composition has been compared to a placebo composition containing only maltodextrin (cryoprotectant) resuspended in 28.75 μL of saline buffer and 1.25 μL of DMSO.

### 2.1 Preparation of the Compositions

15 μg of cryoprotectant has been dissolved in 1.25 μL of DMSO and 28.75 μL of sterile saline buffer to generate one dose of placebo composition.

10 mg of Mifamurtide have been resuspended in 200 μL of DMSO to obtain a 50 μg/μL Mifamurtide solution. 1.25 μL of this solution have been resuspended in 28.75 μL of placebo composition in order to obtain a composition with a final Mifamurtide concentration of 62.5 μg/30 μL (isomolar to 25 μg/30 μL MDP).

### 2.2 Preparation of Animals

Conventional C57B1/6j laboratory mice have been bred in the Gnotobiology Laboratory for more than 10 generations. Mice have been maintained in IVC cages (Tecniplast, Italy), exposed to 12:12 h light-dark cycles, supplied with tap water and fed ad libitum with sterile diet V1124-300 (Ssniff Spezialdiäten GmbH) (irradiated −25 kGy, Bioster, Czech Republic). The mice have been mated and after delivery the litter size was reduced to 6 pups per mother. On day 21, male mice have been weaned on the experimental low-protein, low-fat diet (3.5 kcal/gram, protein: 4.1 wt %, carbohydrate: 78 wt %, fat: 2 wt %) and monitored regularly for up to 56 days after birth. Mice weaned on the low-protein, low-fat diet have been treated 5 times a week with either the placebo composition or the composition comprising mifamurtide as prepared in point 1.1 above. At the end of the experiment, the food and bedding were removed at 8 a.m. and the mice have been sacrificed after 5 hours of fasting by isoflurane inhalation and cervical dislocation. All animals have been sacrificed within one hour between 1 and 2 p.m.

### 2.3 Administration of the Compositions

Each of the compositions has been administered by pipette to the tongue of the mouse. The mouse has been held until the composition had been swallowed completely.

### 2.4 Description of Tests Performed

To measure body length, the mice have briefly been anaesthetised with isoflurane (Piramal Healthcare, UK). The anaesthetised mouse has been held by the tail and the nose-anus length has been measured using a ruler. Relative length has been calculated as % length gain, where body length at day 21 (weaning) has been set to 100%. The length growth rate has been calculated by dividing the absolute length gain from day 21 to day 56 by the number of days (i.e. 35). Femur and tibia bones have been dissected at sacrifice, fixed in 4% paraformaldehyde—PBS overnight at 4° C., washed with PBS and preserved in 70% ethanol. Bone length has been measured using a digital caliper (Festa).

At the end of the experiment, the blood has been collected and left to coagulate at room temperature for 2 hours. Sera have been separated by centrifugation (2,000×g for 5 minutes, 4° C.) and preserved at −80° C. until use. IGF-1 levels have been measured using the Mouse/Rat IGF-1 Quantikine ELISA kit (R&D systems) according to the manufacturer's instructions.

Data have been analysed using the following statistical test: unpaired t-test.

### 2.5 Results

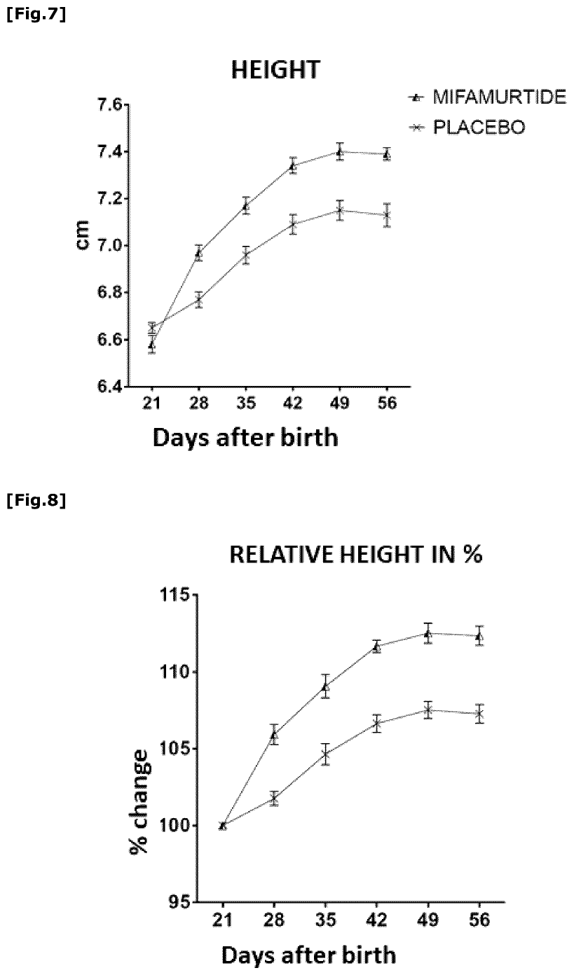

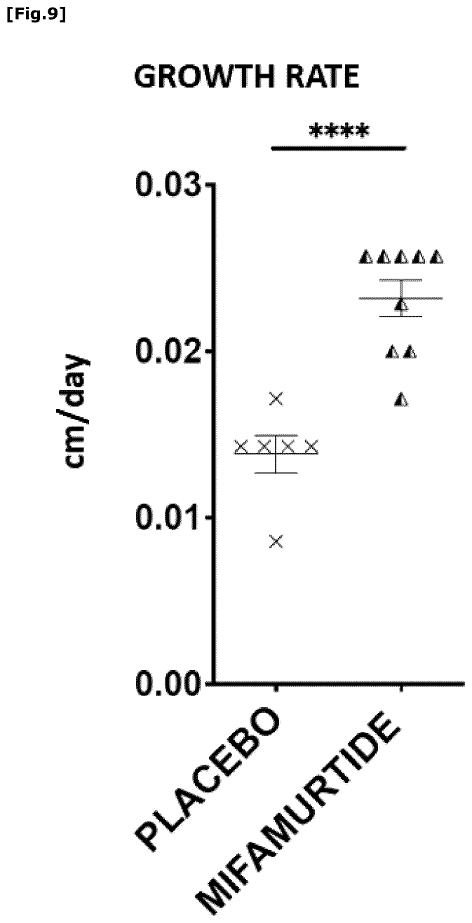

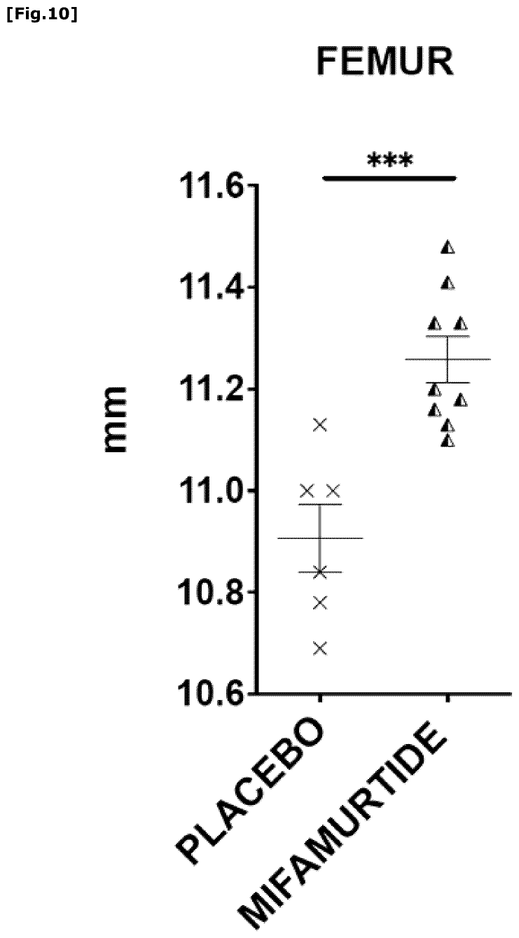

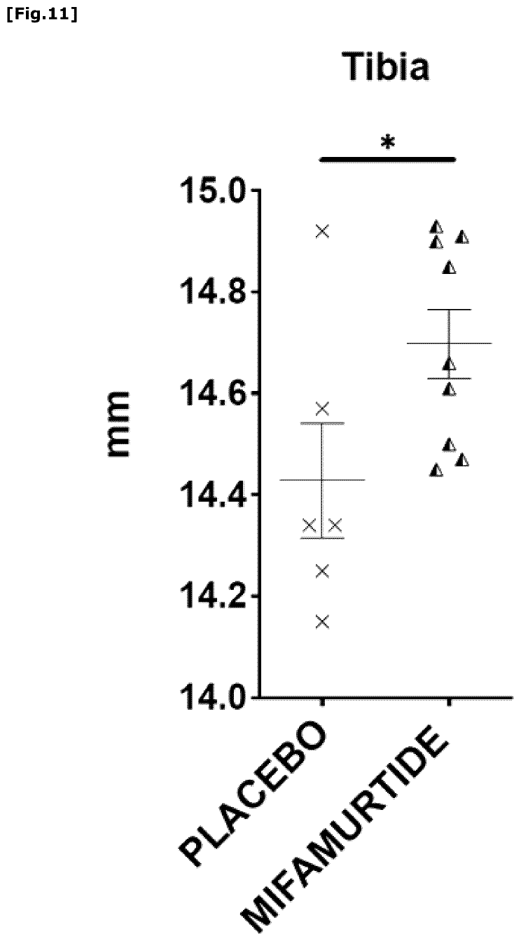

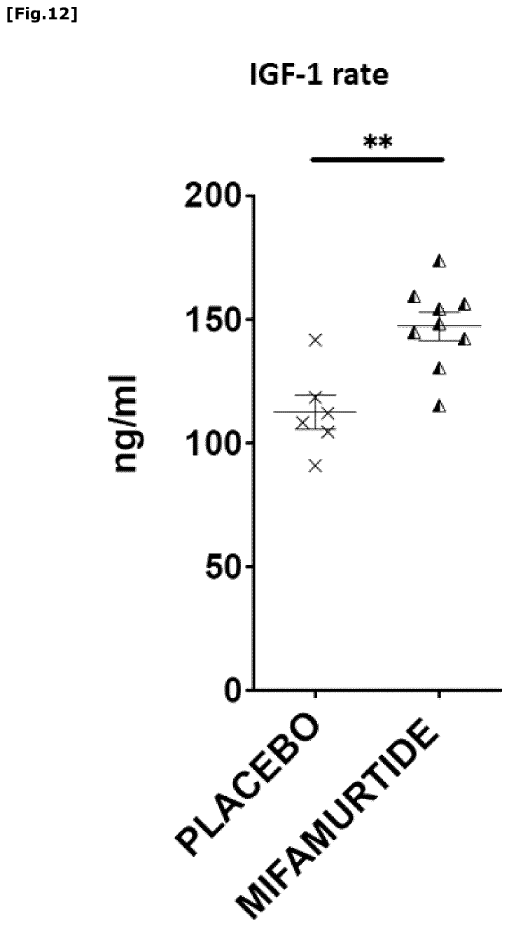

The results are set forth in the appended FIGS. 7 to 12. FIG. 7 represents the course of height (in cm) as a function of the number of days after birth for each of the compositions tested: composition with mifamurtide: curve with empty triangles, placebo composition: curve with crosses. FIG. 8 represents the course of relative growth (% change) as a function of the number of days after birth for each of the compositions tested, using the same symbols as for FIG. 7. FIG. 9 represents the course of the growth rate (in cm/day) for each of the compositions tested. FIG. 10 represents the size of the femur (in mm) for each of the compositions tested. FIG. 11 represents the size of the tibia (in mm) for each of the compositions tested. FIG. 12 represents the IGF blood level (in ng/ml) for each of the compositions tested.

These results indicate a significantly increased relative growth, total height and growth rate in mice given the Mifamurtide-containing composition compared with mice given the placebo composition. The size of the femur, the tibia and the circulating IGF1 level in animals treated with the Mifamurtide-containing composition are significantly increased compared with animals treated with the placebo composition.

### Example 3: Demonstration of the Effects of Combined Administration of MDP and a Bacterium with Intestinal Tropism in a Chronically Undernourished Mouse Model

In this example, the effects of different compositions were tested:
a composition P (placebo) containing only a cryoprotectant (15 μg of maltodextrin) resuspended in 30 μL of sterile saline buffer,
a composition A in accordance with the present invention comprising MDP alone at a rate of 25 μg/day in 30 μL of placebo,
a composition AB in accordance with the present invention comprising MDP at a rate of 25 μg/day and live Lactiplantibacillus plantarum strain WJL bacteria at a rate of 2.10⁸ CFU/day in 30 μL of placebo,
a comparative composition B not forming part of the invention comprising only Lactiplantibacillus plantarum strain WJL bacteria at a rate of 2,108 CFU/day in 30 μL of placebo.

### 3.1 Preparation of the Compositions

Compositions P and A have been prepared as described above in example 1.

Composition AB was prepared in the same way as composition A, but with the addition of 2.10⁸ CFU of the Lactiplantibacillus plantarum strain WJL bacterium to the 30 μL of placebo in addition to the 25 μg of MDP.

Composition B was prepared by adding only 2.10⁸ CFU of Lactiplantibacillus plantarum strain WJL bacterium to the 30 μL of placebo.

### 3.2. Preparation of the Animals

The tests were carried out on male mice with each of the above compositions P, A, AB and B, according to the protocol indicated in example 1, point 1.1 above (56-day period).

### 3.3. Administration of the Compositions

The daily treatment dose was 30 μL of composition P (Placebo) or 25 μg MDP alone in 30 μL of placebo (composition A), or 25 μg MDP and 2.10⁸ CFU of Lactiplantibacillus plantarum strain WJL bacteria in 30 μL of placebo (composition AB) or 2.10⁸ CFU of Lactiplantibacillus plantarum strain WJL bacteria in 30 μL of placebo (composition B).

Each composition has been administered by pipette to the tongue of the mouse. The mouse has been maintained until the composition had been swallowed completely.

### 3.4. Description of Tests Performed

The size in length of the mice has been measured weekly for each batch of mice treated with compositions P, A, AB and B according to the method indicated above in point 1.4 of example 1.

The gain in length growth at D56 (in %) of animals treated with compositions A, AB and B has been calculated relative to the length growth measured at D56 in mice treated with the placebo composition according to the protocol indicated above in point 1.4 of example 1.

The circulating IGF-1 level has been determined for each batch, according to the protocol indicated above in example 1, point 1.4. The percent improvement in the circulating IGF-1 level for each of the compositions tested (A, AB and B) has then been calculated relative to the circulating IGF-1 level measured in mice from the batch treated with the placebo composition (100%).

The numerical data from the measurements have been analysed by appropriate statistical treatments (One way ANOVA with Tukey's multiple comparisons test) in order to test effects of each of the compositions A, AB and B and the possible cumulative effects between treatment with MDP alone or with Lactiplantibacillus plantarum strain WJL bacteria alone or the combination MDP+Lactiplantibacillus plantarum strain WJL bacteria.

### 3.5. Results

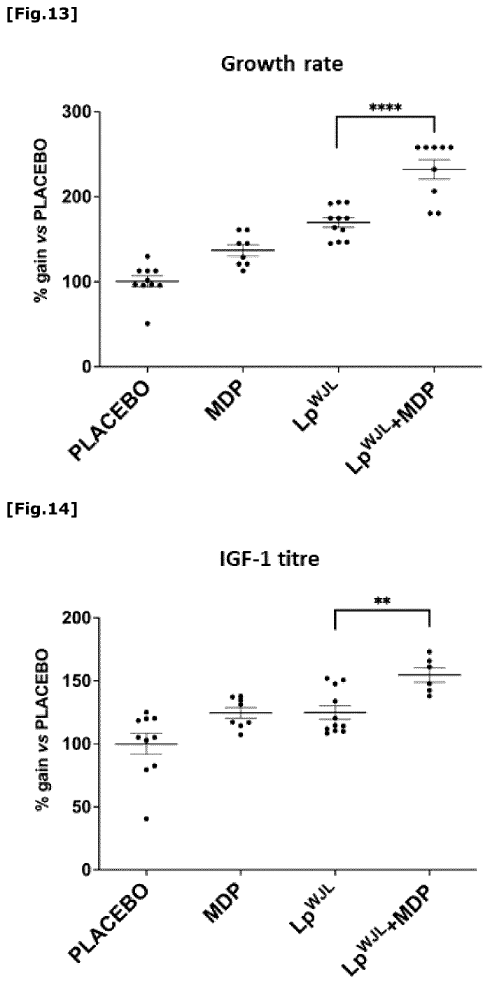

The results obtained are reported in FIGS. 13 and 14.

FIG. 13 represents the % improvement in linear growth for each of the compositions tested in comparison with placebo.

FIG. 14 represents the % improvement in circulating IGF-1 level for each of the compositions tested in comparison with placebo.

These results confirm the positive impact of CDM on the relative linear growth rate with respect to the placebo condition. They also show that the administration of composition AB comprising the combination of MDP and Lactiplantibacillus plantarum strain WJL bacteria significantly increases effect of the treatment on the relative linear growth rate with respect to each of the components administered alone (FIG. 13).

This result is corroborated by the study of circulating IGF-1 level in mice administered the AB composition, i.e. the combination of MPD+Lactiplantibacillus plantarum strain WJL bacteria, for which a significantly more pronounced effect is observed than when each of the components is administered alone.

Consequently, the presence of bacteria with intestinal tropism, such as Lactiplantibacillus plantarum strain WJL bacteria, significantly improves the action of muramyl peptides on skeletal growth.

1-15. (canceled)

16. A composition comprising at least one muramyl peptide, for use in a treatment of skeletal growth retardation in a juvenile human or animal.

17. The composition according to claim 16, wherein said at least one muramyl peptide is selected from muramyl dipeptide and muramyl dipeptide analogues.

18. The composition according to claim 17, wherein the muramyl dipeptide analogues are selected from mifamurtide, murabutide, 6-O-stearoyl-N-acetyl-muramyl-L-alanyl-D-isoglutamine, muropeptide MurNAc-Ala-D-isoGln-L, and mixtures thereof.

19. The composition according to claim 16, wherein said at least one muramyl peptide is muramyl dipeptide.

20. The composition according to claim 16, wherein said at least one muramyl peptide represents from 10 μg to 100 mg per unit dose of composition.

21. The composition according to claim 16, further comprising at least one adjuvant for promoting growth.

22. The composition according to claim 21, wherein said adjuvant is selected from strains with intestinal tropism, bacterial constituents, postbiotics and mixtures thereof.

23. The composition according to claim 21, wherein said adjuvant is selected from bacteria belonging to the Lactobacilluseae, Streptoccaceae, Enterococcaceae, Leuconostocaceae and Bifidobacteriaceae families.

24. The composition according to claim 22, wherein said strains are selected from the Lactobacillus plantarum, Lactobacillus fermentum and Lactobacillus casei species.

25. The composition according to claim 22, wherein said strains are selected from L. plantarum WJL, L. plantarum IGFL1, L. plantarum IGFL2, L. plantarum G821, L. plantarum NIZ02877, L. casei ATCC 393, L. casei L919, L. paracasei ATCC25302, L. paracasei Shirota, L. fermentum ATCC9338, L. rhamnosus L900, L. rhamnosus L908, L. rhamnosus GG, and mixtures thereof.

26. The composition according to claim 22, wherein the bacterial constituents are selected from inactivated bacteria, bacterial lysates, purified bacterial walls and purified bacterial wall fragments of bacteria with intestinal tropism, peptidoglycans and teichoic acids.

27. The composition according to claim 16, further comprising, as an additional ingredient, one or more food supplements and/or nutrients selected from carbohydrates, lipids, peptides other than muramyl peptides, proteins, fatty acids, amino acids, vitamins, minerals and prebiotics.

28. A method for treating skeletal growth retardation in a juvenile human or animal subject, said method comprising administering a composition comprising at least one muramyl peptide to a subject in need thereof.

29. The treatment method according to claim 28, wherein the subject is a chronically undernourished human or animal subject due to protein deficiency.

30. A probiotic treatment method for promoting skeletal growth in an animal or human subject, said method comprising administering to said subject a composition comprising at least one muramyl peptide and at least one bacterium having intestinal tropism as an adjuvant, said bacterium being alive or inactivated.
